(19) United States
(12) Patent Application Publication
(10) Pub. No.: US 2026/0188133 A1
(43) Pub. Date: Jul. 2, 2026

(54) DIGITAL PLATFORM FOR LEARNING AND SHARING SURGICAL SKILLS

(71) Applicant: Kaushal Kumar PATHAK, Udupi (IN)

(72) Inventors: Kaushal Kumar PATHAK, Udupi (IN); Ashish MISHRA, Kanpur (IN); Ayush Kumar PATHAK, Delhi (IN)

(73) Assignee: Kaushal Kumar PATHAK, Udupi (IN)

(21) Appl. No.: 19/125,812

(22) PCT Filed: Jul. 21, 2023
(86) PCT No.: PCT/IB2023/057452

§ 371 Date: Apr. 30, 2025

(30) Foreign Application Priority Data
Oct. 31, 2022 (IN) 202241062043
Jan. 30, 2023 (IN) 202341006003
Jul. 21, 2023 (WO) PCT/IB2023/057452

(51) Int. Cl. G09B 7/00; A61B 90/00; G09B 5/06; G16H 40/67; G16H 70/20; G16H 80/00

(52) U.S. Cl. CPC G09B 7/00; A61B 90/361; G16H 40/67; G16H 70/20; G16H 80/00; A61B 2090/373; G09B 5/065

(57) ABSTRACT

Aspects of the present disclosure provides a digital platform for learning and sharing surgical skills by surgical practitioners (e.g., senior surgeons, junior surgeons). In one embodiment, a system acquires a multimedia stream of a first surgical practitioner (e.g., senior surgeon) performing a surgical operation, stores the multimedia stream in a non-volatile storage and shares a portion of the multimedia stream with one or more surgical practitioners while the surgical operation is being performed. Upon receiving, from a second surgical practitioner (e.g., junior surgeon), questions related to the surgical operation, the system enables the first surgical practitioner to provide answers to the questions while the surgical operation is being performed.

### PRIORITY CLAIM

The instant patent application is related to and claims priority from the co-pending India provisional patent applications (1) Serial No.: 202241062043, Filed: 31 Oct. 2022, entitled, “DIGITAL PLATFORM FOR LEARNING AND SHARING SURGICAL SKILLS” and (2) Serial No.: 202341006003, Filed: 30 Jan. 2023, entitled, “METHOD COMPRISING TELEMENTORSHIP IN LAPROSCOPIC SURGERY” which are incorporated in their entirety herewith.

### BACKGROUND OF THE INVENTION

### Technical Field

The present disclosure relates to digital healthcare management and more specifically to a digital platform for learning and sharing surgical skills. The digital platform also facilitates telementorship.

### Related Art

Modern surgical practice is complex and highly specialized. The cornerstone of safe and successful surgery is the acquisition of specialist knowledge and skills, built on a solid foundation of early years training. Traditionally training was delivered over many years wholly based in the workplace, supplemented by textbook learning.

E-learning has rapidly become a ‘must-do’ across Higher Education (HE). Early efforts in e-learning consisted mainly of loading lecture notes and slides onto a website. This would now be regarded as resource distribution rather than e-learning and it failed to involve active teaching. E-learning is now characterized by virtual learning environments (VLE), usually operating within a managed learning environment administering all aspects of the course through a standard interface. In recent years e-learning and VLEs have become so commonplace in undergraduate medical courses that current trainees are already experienced users.

However, highly literate in e-learning, medical graduates now emerge from a broad-based curriculum and intending surgeons do so with insufficient basic science knowledge. Early years trainees thus carry a huge burden of study and skills acquisition, disrupted by mainly on-call service commitments.

Therefore, there is a need to provide a single digital platform for learning and sharing surgical skills. More particularly, there is need for a digital platform that facilitates senior surgeons to share their knowledge and skills with junior surgeons.

### SUMMARY OF THE INVENTION

Aspects of the present disclosure provides a digital platform for learning and sharing surgical skills by surgical practitioners (e.g., senior surgeons, junior surgeons). In one embodiment, a system acquires a multimedia stream of a first surgical practitioner (e.g., senior surgeon) performing a surgical operation, stores the multimedia stream in a non-volatile storage and shares a portion of the multimedia stream with one or more surgical practitioners while the surgical operation is being performed. Upon receiving, from a second surgical practitioner (e.g., junior surgeon), questions related to the surgical operation, the system enables the first surgical practitioner to provide answers to the questions while the surgical operation is being performed.

According to another aspect of the present disclosure, the acquiring (noted above) comprises displaying on an operation theater (OT) monitor, a video of the performance of the surgical operation captured using one or more cameras, recording the video being displayed on the OT monitor and converting the video to the multimedia stream. The sharing comprises streaming the portion of the multimedia stream via a network.

According to one more aspect of the present disclosure, the system facilitates uploading, by the first surgical practitioner, portions of the multimedia stream as corresponding videos and facilitates the second surgical practitioner to view the corresponding videos. In one embodiment, each video is associated with one or more of tutorials, research papers and publications.

According to yet another aspect of the present disclosure, the system facilitates posting, by the first surgical practitioner prior to the surgical operation, an observership course. Upon determining that a set of surgical practitioners have registered for the observership course, the system includes the set of surgical practitioners in the one or more surgical practitioners whereby the set of surgical practitioners are enabled to observe and interact with the first surgical practitioner while the surgical operation is being performed.

According to an aspect of the present disclosure, the system creates a group of surgical practitioners and shares the portion of the multimedia stream with the group of surgical practitioners while the surgical operation is being performed.

According to another aspect of the present disclosure, a digital platform for surgical practitioners comprises an acquisition system to acquire a multimedia stream of a first surgical practitioner performing a surgical operation and to store the multimedia stream in a non-volatile storage, a streaming server to share a portion of the multimedia stream to one or more surgical practitioners while the surgical operation is being performed and an interface system to receive from a second surgical practitioner, questions related to the surgical operation and to enable the first practitioner to provide answers to the questions while the surgical operation is being performed.

According to one more aspect of the present disclosure, the acquisition system comprises an operation theater (OT) monitor to display a video of the performance of the surgical operation captured using one or more cameras, a recorder to record the video being displayed on the OT monitor and a converter to convert the video to the multimedia stream. To share, the streaming server streams the portion of the multimedia stream via a network.

An aspect of the present disclosure provides mentorship to surgical practitioners. In one embodiment, a digital processing system sends, by a first surgical practitioner (e.g., junior surgeon) to a second surgical practitioner (e.g., senior surgeon), a request for mentorship for a surgical operation. Upon receiving, from the second surgical practitioner, a confirmation of mentorship in response to the request, the system acquires a multimedia stream of the first surgical practitioner performing the surgical operation, stores the multimedia stream in a non-volatile storage and shares a portion of the multimedia stream with the second surgical practitioner while the surgical operation is being performed. Upon receiving, from the first surgical practitioner, questions related to the surgical operation, the system enables the second practitioner to provide answers to the questions while the surgical operation is being performed. As such, the second surgical practitioner is enabled to provide assistance to the first surgical practitioner while the surgical operation is being performed. In one embodiment, the surgical operation is laparoscopic surgery.

According to another aspect of the present disclosure, the system maintains the details of one or more surgical practitioners and facilitates the first surgical practitioner to select the second surgical practitioner from the one or more surgical practitioners.

Thus, aspects of the present disclosure provide for a single digital platform on which senior surgeons can give all around training to junior surgeons to develop a greater number of expert surgeons. The senior surgeons are enabled to take virtual classes for junior surgeons, The senior surgeons can show live surgery (surgical operation) and explain to junior surgeons all the steps and clear doubt during live surgery. The senior surgeons can post surgery videos and tutorial materials so that junior surgeons can refer to the videos as per their convenience. Since online support may not be sufficient for junior surgeons to grab surgical skills completely, senior surgeons can post observership courses and hands on training course, in which the senior surgeons will teach limited junior surgeons inside the OT physically.

Even then, if junior surgeons are not very confident while doing particular surgery for the first time, the junior surgeons can find a mentor of his/her choice for their upcoming surgery. Junior surgeons can locate senior surgeons using the platform and take online classes. Surgeons may also refer his/her patients to any other surgeons of any specialty, anywhere, based on availability of surgeons, which will help the patient to save time and money.

Several aspects of the invention are described below with reference to examples for illustration. However, one skilled in the relevant art will recognize that the invention can be practiced without one or more of the specific details or with other methods, components, materials and so forth. In other instances, well-known structures, materials, or operations are not shown in detail to avoid obscuring the features of the invention. Furthermore, the features/aspects described can be practiced in various combinations, though only some of the combinations are described herein for conciseness.

### BRIEF DESCRIPTION OF THE DRAWINGS

Example embodiments of the present invention will be described with reference to the accompanying drawings briefly described below.

FIG. 1 is a block diagram illustrating an example computing system in which various aspects of the present invention can be implemented.

FIG. 2 is a flow chart illustrating the manner in which a digital platform for learning and sharing surgical skills is provided according to aspects of the present invention.

FIG. 3 is a block diagram illustrating the manner in which a multimedia stream of a surgical practitioner performing a surgical operation is acquired in one embodiment.

FIG. 4 illustrates a dashboard user interface provided to surgical practitioners in one embodiment.

FIG. 5A illustrates a user interface using which a surgical practitioner is facilitated to organize a live surgery to be streamed in one embodiment.

FIG. 5B illustrates a user interface using which a surgical practitioner is facilitated to attend streaming live surgeries in one embodiment.

FIG. 5C illustrates a user interface of live streaming of a surgical operation in progress in one embodiment.

FIG. 5D illustrates a user interface using which a surgical practitioner is facilitated to post/upload a video of a surgery in one embodiment.

FIG. 5E illustrates a user interface using which a surgical practitioner is facilitated to view posted/uploaded surgery videos in one embodiment.

FIG. 5F illustrates a user interface using which a surgical practitioner is facilitated to post an observership course in one embodiment.

FIG. 5G illustrates a user interface using which a surgical practitioner is facilitated to view (and register with desired ones of) posted observership courses in one embodiment.

FIG. 5H illustrates a user interface using which a surgical practitioner is facilitated to view/access the various associations and/or groups to which he/she belongs in one embodiment.

FIG. 5I illustrates a user interface using which a surgical practitioner is facilitated to create a new association/group in one embodiment.

FIG. 6 is a flow chart illustrating the manner in which mentorship to surgical practitioners is provided according to aspects of the present invention.

FIG. 7A illustrates a user interface using which a surgical practitioner is facilitated to request a mentorship in one embodiment.

FIG. 7B illustrates a user interface using which a surgical practitioner is facilitated to request a mentorship in another embodiment.

FIG. 7C illustrates a user interface of live streaming of a surgical operation in progress as part of mentorship in one embodiment.

FIG. 8 is a block diagram illustrating the details of a digital processing system in which various aspects of the present invention are operative by execution of appropriate execution modules.

In the drawings, like reference numbers generally indicate identical, functionally similar, and/or structurally similar elements. The drawing in which an element first appears is indicated by the leftmost digit(s) in the corresponding reference number.

### DETAILED DESCRIPTION OF THE INVENTION

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

Reference throughout this specification to “one embodiment”, “an embodiment”, or similar language means that a particular feature, structure, or characteristic described in connection with the embodiment is included in at least one embodiment of the present invention. Thus, appearances of the phrases “in one embodiment”, “in an embodiment” and similar language throughout this specification may, but do not necessarily, all refer to the same embodiment.

The use of “including”, “comprising”, or “having” and variations there of herein is meant to encompass the items listed thereafter and equivalents thereof as well as additional items. The terms “a” and “an” herein do not denote a limitation of quantity, but rather denote the presence of at least one of the referenced items. Further, the use of terms “first”, “second”, and “third”, and the like, herein do not denote any order, quantity, or importance, but rather are used to distinguish one element from another.

As used herein, the singular forms “a”, “an”, and “the” include both singular and plural referents unless the context clearly dictates otherwise. By way of example, “a dosage” refers to one or more than one dosage. The terms “comprising”, “comprises” and “comprised of” as used herein are synonymous with “including”, “includes” or “containing”, “contains”, and are inclusive or open-ended and do not exclude additional, non-recited members, elements, or method steps.

All documents cited in the present specification are hereby incorporated by reference in their totality. In particular, the teachings of all documents herein specifically referred to are incorporated by reference.

Example embodiments of the present invention are described with reference to the accompanying figures.

### 1. Definitions

Digital platform—An application based on a combination of hardware and software erected on present day cloud technology that makes the evolution of hardware or software easy.

Surgery—Medical treatment (operation) in which your body is cut open so that part of it can be removed or repaired.

Surgeon—A doctor who is specially trained to perform medical/surgical operations.

Observership course—Surgical training in which junior surgeons observe the surgery of senior surgeons from inside the OT, so that they can observe all the unique surgical technique of the senior surgeons unlike edited videos.

Mentorship—Junior surgeons requesting senior surgeons to be present during the conduct of their surgery so that the senior surgeons can provide surgical training and assistance during the surgery.

Telementorship—Mentorship when the senior surgeon is present virtually (via an audio-video call), and not physically. In the following disclosure, the terms “mentorship” and “telementorship” are used interchangeably.

### 2. Example Environment

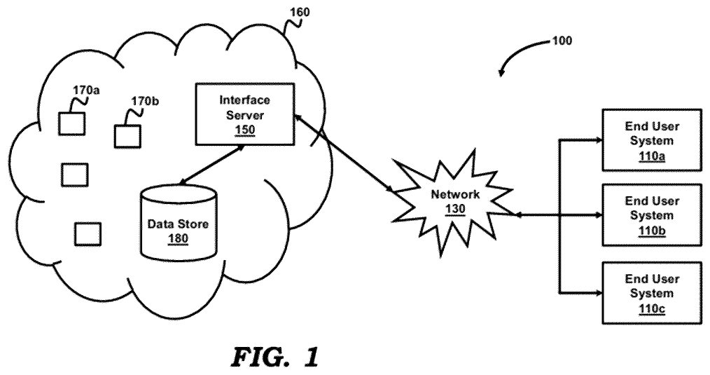

FIG. 1 is a block diagram illustrating an example computing system (100) in which various aspects of the present invention can be implemented. The block diagram is shown containing end user systems 110a-110c, network 130, and cloud 160 (which in turn is shown containing a number of nodes such as node 170a and 170b, interface server 150 and data store 180).

Merely for illustration, only representative number/type of systems is shown in FIG. 1. Many computing systems often contain many more systems, both in number and type, depending on the purpose for which the computing system is designed. Each stem/device of FIG. 1 is described below in further detail.

Network 130 provides connectivity between end user systems 110a-110c, and nodes of cloud 160 (such as node 170a/170b, interface server 150 and data store 180). Network 130 may represent Wireless/LAN networks implemented using protocols such as Transport Control Protocol/Internet Protocol (TCP/IP), or circuit switched network implemented using protocols such as GSM, CDMA, etc. as is well known in the relevant arts.

In general, network 130 provides transport of packets, with each packet containing a source address (as assigned to the specific system from which the packet originates) and a destination address, equaling the specific address assigned to the specific system to which a packet is destined/targeted. The packets would generally contain the requests and responses between the various systems connected via network 130 as described in detail in the below sections.

Each of end user systems 110a-110c represents a system such as a personal computer, workstation, mobile phone (e.g., iPhone available from Apple Corporation), tablet, portable device (also referred to as “smart” “devices”) that operate with a generic operating system such as Android operating system available from Google Corporation, etc., used by users to send (user) requests to nodes of cloud 160 such as interface server 150. In addition, each of end user systems 110a-110c may include various hardware (and corresponding software) sensors such as camera, microphone, accelerometers, etc. In general, an end user system enables a user to send user requests for performing desired tasks to interface server 150 and to receive corresponding responses containing the results of performance of the requested tasks.

Cloud 160 is a collection of nodes (such as node 170a/170b) that may include processing nodes, connectivity infrastructure, data storages, administration systems, etc., which are engineered to together host software applications. Cloud 160 may be provided on a public cloud infrastructure (such as Amazon Web Services (AWS) available from Amazon.com, Inc., Google Cloud Platform (GCP) available from Google LLC, etc.) that provides a virtual computing infrastructure for various customers, with the scale of such computing infrastructure being specified often on demand. Alternatively, cloud 160 may be provided on an enterprise system (or a part thereof) on the premises of the business organizations (referred to as an “on-prem” cloud). Cloud 160 may also be a “hybrid” infrastructure containing some nodes of a public cloud infrastructure and other nodes of an enterprise system.

It may be appreciated that each of interface server 150 and data store 180 are implemented on corresponding nodes of cloud 160. Some of the other nodes (such as node 170a/170b) of cloud 160 may be implemented as corresponding data stores similar to data store 180, while other nodes of the cloud 160 may be implemented as corresponding server systems, similar to interface server 150.

Data store 180 represents a non-volatile storage, facilitating storage and retrieval of a collection of data by interface server 150. A data store may maintain information such as user data received from end user systems 110a-110c, data related to performance of tasks noted above, etc. In one embodiment, a data store is implemented using relational database technologies where the data is maintained in the form of databases containing tables and columns and provides storage and retrieval of data using structured queries such as SQL (Structured Query Language), as is well known in the relevant arts. Alternatively, a data store may be implemented as a file server and store data in the form of one or more files organized in the form of a hierarchy of directories, as is well known in the relevant arts.

Interface server 150 represents a system, such as a web and/or application server, executing various applications designed to perform one or more tasks requested from end user systems. For example, each of the servers may execute one or more healthcare applications. Each server may perform the tasks using data maintained internally in the server, on external data (e.g., maintained in data store 180) or on data received as part of the requests (e.g., data received from end user systems 110a-110c). Each server may also send the results of performance of the tasks to end user systems 110a-110c or one or more nodes of cloud 160. Furthermore, each server may maintain some of the received information (such as the data from end user systems 110a-110c) and the result of performance of the tasks in data store 180.

According to an aspect, interface server 150 along with data store 180 (and other nodes of cloud 160) together provide a digital platform for learning and sharing surgical skills by surgical practitioners (e.g., senior surgeons, junior surgeons). The manner in which interface server 150 provides several features of the present invention is described below with examples.

### 2. Providing a Digital Platform for Learning and Sharing Surgical Skills

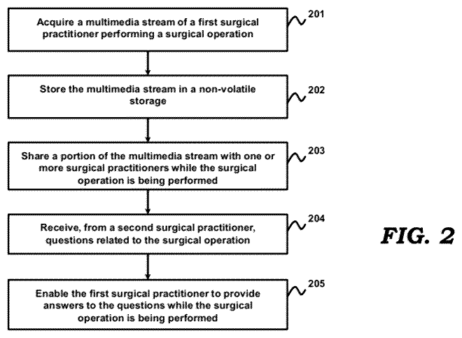

FIG. 2 is a flow chart illustrating the manner in which a digital platform for learning and sharing surgical skills is provided according to aspects of the present invention. The flowchart is described with respect to FIG. 1, in particular, interface server 150, merely for illustration. However, various features can be implemented in other systems and/or other environments also without departing from the scope of various aspects of the present invention, as will be apparent to one skilled in the relevant arts by reading the disclosure provided herein.

In addition, some of the steps may be performed in a different sequence than that depicted below, as suited in the specific environment, as will be apparent to one skilled in the relevant arts. Many of such implementations are contemplated to be covered by several aspects of the present invention.

In step 201, interface server 150 acquire a multimedia stream of a first surgical practitioner (e.g., senior surgeon) performing a surgical operation. The multimedia stream may be acquired in a known way.

According to an aspect, interface server 150 acquires the multimedia stream by displaying on an operation theater (OT) monitor, a video of the performance of the surgical operation captured using one or more cameras, recording the video being displayed on the OT monitor and converting the video to the multimedia stream.

In step 202, interface server 150 stores the multimedia stream in a non-volatile storage such as data store 180. The multimedia stream may be stored in any desirable format as will be apparent to one skilled in the arts.

In step 203, interface server 150 shares a portion of the multimedia stream with one or more surgical practitioners while the surgical operation is being performed. Such sharing may entail streaming the portion of the multimedia stream via a network (such as 130) to one or more end user systems 110a-110c used by the one or more surgical practitioners. The one or more surgical practitioners to whom the multimedia stream is streamed may be determined in a known way.

According to an aspect, interface server 150 facilitates posting, by the first surgical practitioner prior to the surgical operation, an observership course. Upon determining that a set of surgical practitioners have registered for the observership course, interface server 150 includes the set of surgical practitioners in the one or more surgical practitioners whereby the set of surgical practitioners are enabled to observe and interact with the first surgical practitioner while the surgical operation is being performed.

According to another aspect, interface server 150 creates a group of surgical practitioners and shares the portion of the multimedia stream with the group of surgical practitioners while the surgical operation is being performed.

In step 204, interface server 150 receives, from a second surgical practitioner (e.g., junior surgeon), questions related to the surgical operation. The questions may be received as text, audio, or video or a combination thereof.

In step 205, interface server 150 enables the first surgical practitioner to provide answers to the questions while the surgical operation is being performed. For example, questions received as text may be displayed to the first surgical practitioner while the surgical operation is being performed, with the first surgical practitioner then providing the answers via audio, which are then provided to the second surgical practitioner.

Thus, interface server 150 (along with data store 180) provides a single digital platform on which senior surgeons can show live surgery (surgical operation) and explain to junior surgeons all the steps and clear doubt during live surgery. The senior surgeons are enabled to take virtual classes for junior surgeons.

According to an aspect, interface server 150 also facilitates uploading, by the first surgical practitioner, portions of the multimedia stream as corresponding videos and facilitates the second surgical practitioner to view the corresponding videos. In one embodiment, each video is associated with one or more of tutorials, research papers and publications.

The manner in which interface server 150 provides aspects of the present invention according to the operation of FIG. 2 is described below with examples.

### 3. Example Digital Platform

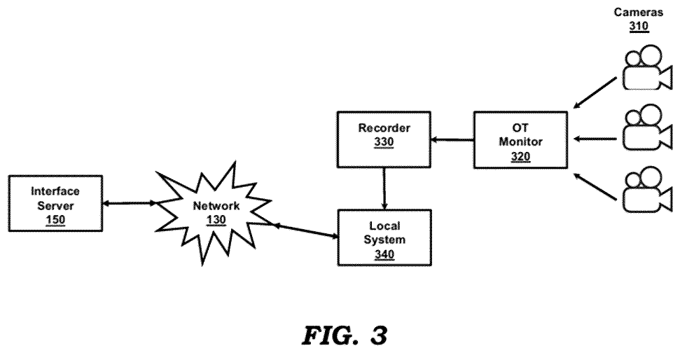

FIGS. 3, 4 and 5A-5I together illustrate the manner in which a digital platform for learning and sharing surgical skills is provided in one embodiment. Each of the Figures is described in detail below.

FIG. 3 is a block diagram illustrating the manner in which a multimedia stream of a surgical practitioner performing a surgical operation is acquired in one embodiment. The block diagram is shown containing cameras 310, OT (operating theater) monitor 320, recorder 330, and local system 340. Each of the blocks is described in detail below.

Cameras 310 capture the performance of the surgical operation from different angles and/or viewpoints as corresponding videos. Cameras 310 may be standard equipment or may be specialized equipment used specifically in operating theatres for capturing surgical operations.

OT monitor 320 is a display unit that displays the videos captured by cameras 310. It may be appreciated that the videos from all of cameras 310 are received by OT monitor 320 though only some of the videos may be displayed on the display unit at any given time instance.

Recorder 330 records the videos received by OT monitor 320. In one embodiment, recorder 330 is a High-Definition Multimedia Interface (HDMI) video capturing device that is connected between OT monitor 320 and local system 340. Recorder 330 captures all the videos received by OT monitor 320 from cameras 310.

Local system 340 represents a system such as a laptop that receives the videos captured by recorder 330 and converts the received videos to one or more multimedia streams. Local system 340 may execute specialized software such as OBS (Open Broadcaster Software) Studio available from “https://obsproject.com/” for such conversion. As is well known, OBS Studio is a free and open-source software for video recording and live streaming. OBS studio helps to capture the same surgery video displayed on OT monitor 320 in local system 340.

Local system 340 thereafter uploads the multimedia stream to interface server 150, which then shares the live surgery streams to one or more surgical practitioners. In an alternative embodiment, interface server 150 may stream the multimedia streams using third party servers (not shown) such as Twitch™, Youtube™, etc. well known in the arts.

Other features of the present invention provided by interface server 150 are described below with examples.

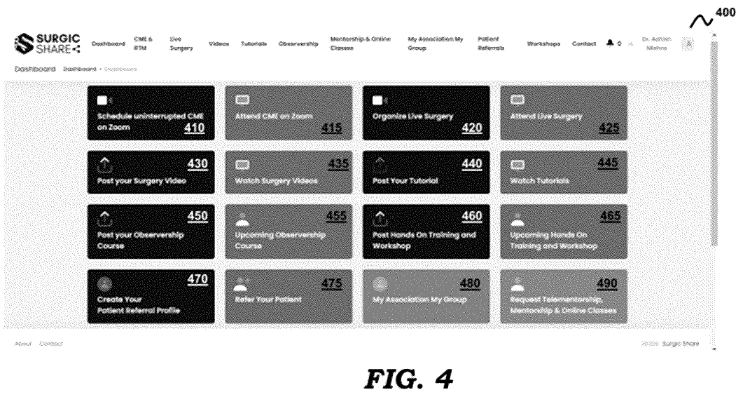

FIG. 4 illustrates a dashboard user interface provided to surgical practitioners in one embodiment. The user interface (and also other user interfaces described below) may be provided as corresponding web pages by interface server 150 in response to users specifying corresponding URLs (universal resource locators) in a browser executing in end user systems 110a-110c. The user interfaces may be displayed on a display unit (not shown in the FIGS.) associated with end user systems 110a-110c.

Display area 400 depicts various features made available to surgical practitioners according to several aspects of the present invention. A surgical practitioner may select the desired feature/option shown in display area 400 to access the corresponding functionality provided by interface server 150.

Option 410 “Schedule uninterrupted CME on Zoom” facilitates a (senior) surgeon to schedule a CME (continuing medical education) lecture/meeting via video conferencing, while option 415 “Attend CME on Zoom” facilitates a (junior) surgeon/student to attend the scheduled CME lecture/class. Every surgeons gets mail and notification to attend the CME/meeting at the scheduled time.

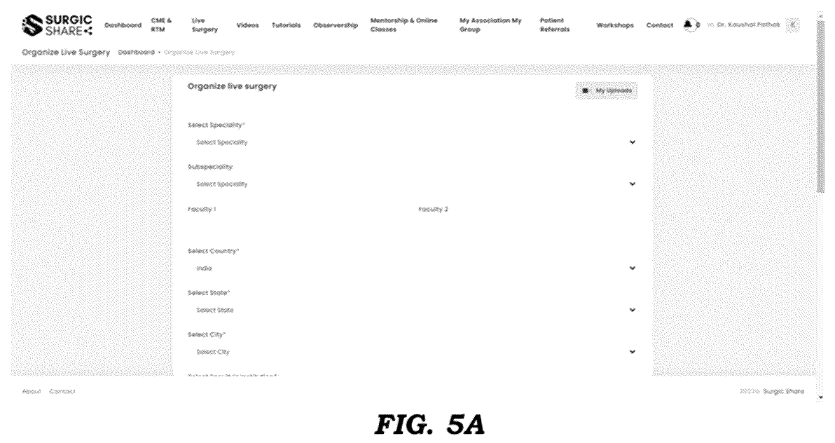

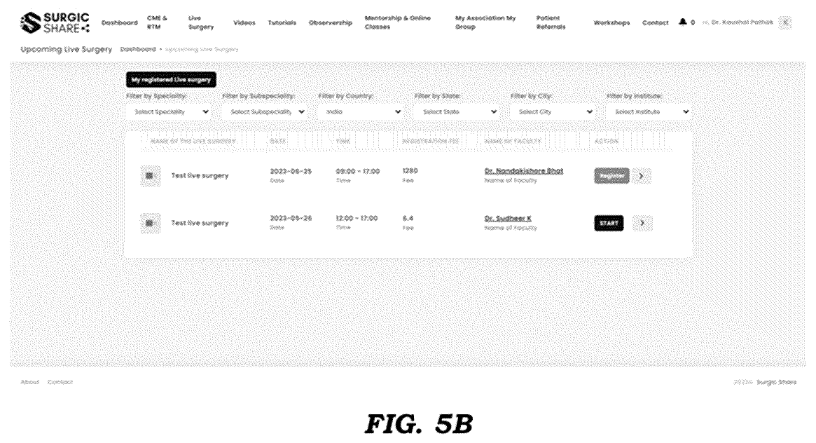

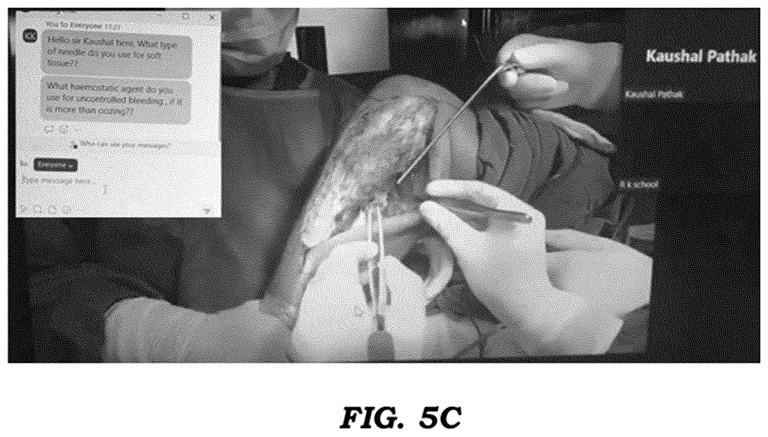

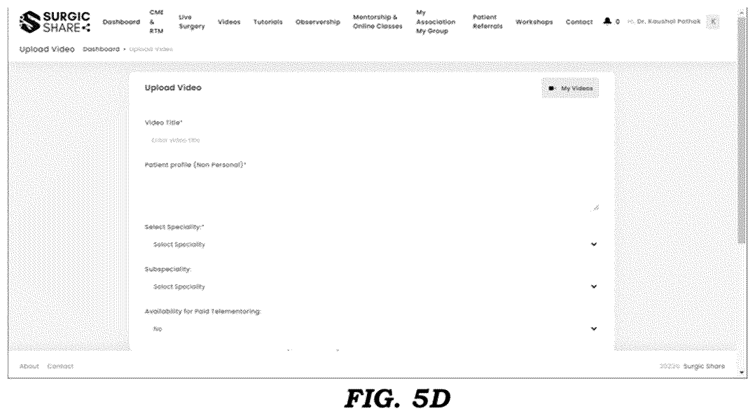

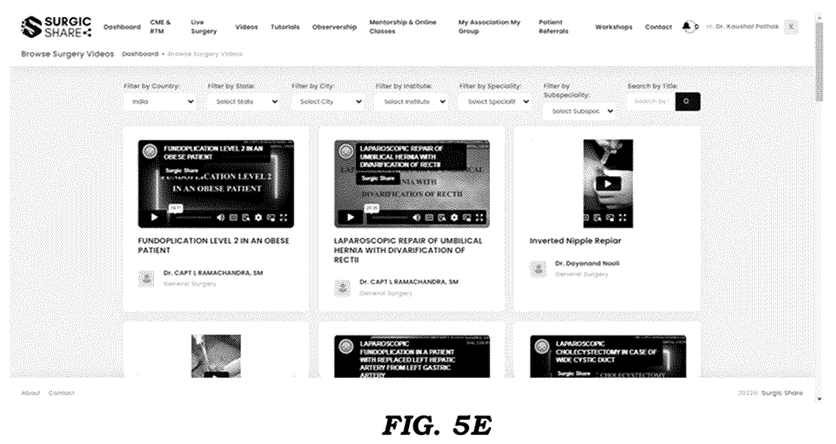

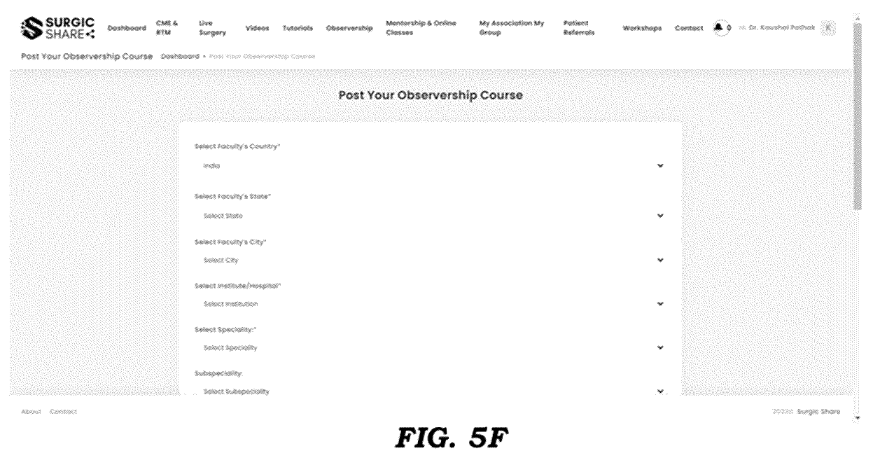

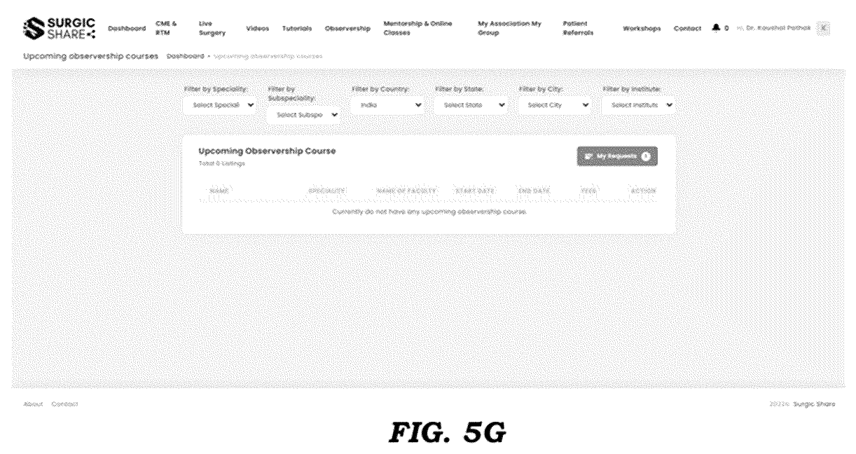

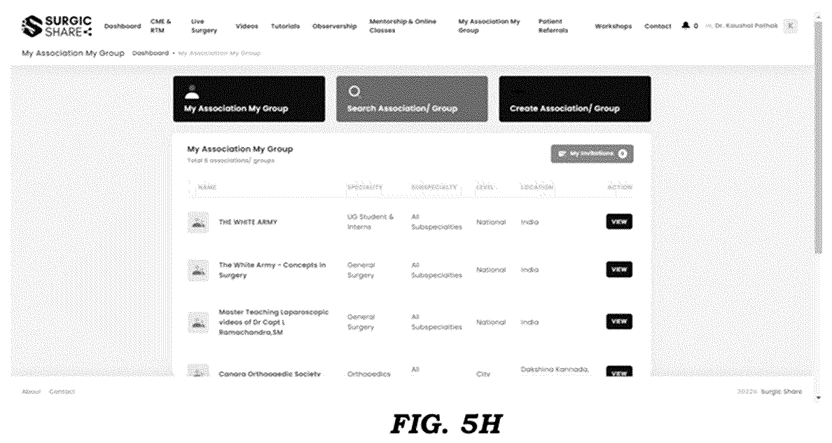

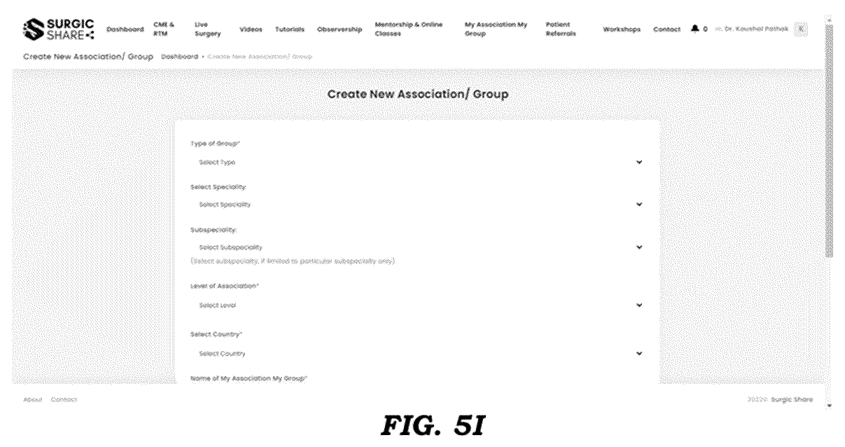

Option 420 “Organize Live Surgery” facilitates a (senior) surgeon to schedule a surgery that is sought to be streamed, while option 425 “Attend Live Surgery” facilitates a (junior) surgeon/student to register and attend scheduled surgeries of interest. FIG. 5A illustrates a user interface using which a surgical practitioner is facilitated to organize a live surgery to be streamed in one embodiment. FIG. 5B illustrates a user interface using which a surgical practitioner is facilitated to attend streaming live surgeries in one embodiment.

FIG. 5C illustrates a user interface of live streaming of a surgical operation in progress in one embodiment. It may be observed that that a text chat has been enabled between a surgical practitioner performing the surgery and a surgical practitioner viewing the surgery. By using the user interfaces of FIGS. 5A-5C, senior surgeons can schedule their upcoming live surgeries from OT and individual surgeons can watch live surgery sitting at home.

Referring again to FIG. 4, option 430 “Post your Surgery Video” facilitates a (senior) surgeon to post/upload a video of a surgery, while option 435 “Watch Surgery Videos” facilitates a (junior) surgeon/student to view the posted/uploaded surgery videos. FIG. 5D illustrates a user interface using which a surgical practitioner is facilitated to post/upload a video of a surgery in one embodiment. FIG. 5E illustrates a user interface using which a surgical practitioner is facilitated to view posted/uploaded surgery videos in one embodiment.

Referring again to FIG. 4, option 440 “Post Your Tutorial” facilitates a (senior) surgeon to post/upload a tutorial, while option 445 “Watch Tutorials” facilitates a (junior) surgeon/student to view the posted/uploaded tutorials. In one embodiment, user interfaces similar to FIGS. 5D and 5E are provided for posting/viewing tutorials. It may be appreciated that by using the interfaces of FIGS. 5D and 5E, senior surgeons can post their surgery videos and tutorials of even more than two hours duration along with provision for adding research paper, and publications. The junior surgeons can watch surgery videos and tutorials as per their convenience.

In another embodiment, senior surgeons can post their surgery videos to the rest of the world. Junior surgeons from rest of the world can see his/her surgery videos and can request for paid mentorship, observership, online classes, etc. with the senior surgeons, to clarify his/her doubt, confusion or to get the confidence required.

Option 450 “Post your Observership Course” facilitates a (senior) surgeon to post an observership course, while option 455 “Upcoming Observership Courses” facilitates a (junior) surgeon/student to view and register for the posted observership courses. FIG. 5F illustrates a user interface using which a surgical practitioner is facilitated to post an observership course in one embodiment. FIG. 5G illustrates a user interface using which a surgical practitioner is facilitated to view (and register with desired ones of) posted observership courses in one embodiment. By using the user interfaces of FIGS. 5F and 5G, senior surgeons may offer various observership courses, wherein he/she mention details of observership course including fees and number of slots available. Interested surgeons can register and attend his/her surgery inside his/her OT and learn and take first-hand experience to grab surgical skills from top surgeons.

Referring again to FIG. 4, option 460 “Post Conference, Workshop, Hands on Training” facilitates a (senior) surgeon to post conferences, workshops, and hands on training sessions, while option 465 “Upcoming Conference, Workshop, Hands on Training” facilitates a (junior) surgeon/student to view and register for the posted conferences, workshops, and hands on training sessions. In one embodiment, user interfaces similar to FIGS. 5F and 5G are provided for posting/viewing conferences, workshops, and hands on training sessions. Again, hands on training course can be provided by senior surgeons, wherein he/she mentions details of the hands on training course. Interested surgeons can register and attend hands on training course and improve surgical skills.

Option 470 “Create Your Patient Referral Profile” facilitates a surgeon to create his/her profile that would be sent as part of patient referral, while option 475 “Refer Your Patient” facilitates a surgeon refer a patient to another surgeon registered with the digital platform. Option 480 “My Association My Group” facilitates a surgeon to view/access the various associations and/or groups to which he/she belongs (where he/she had previously registered/joined), and also to create new Association/Group. FIG. 5H illustrates a user interface using which a surgical practitioner is facilitated to view/access the various associations and/or groups to which he/she belongs in one embodiment. FIG. 5I illustrates a user interface using which a surgical practitioner is facilitated to create a new association/group in one embodiment. In one embodiment, a surgeon can create a closed group of thousands of surgeons of same specialty with their mobile number or email, within minutes.

Referring again to FIG. 4, option 490 “Telementorship, Mentorship & Online Classes” facilitates a surgeon to request for a telementorship/mentorship from other surgeons registered with the digital platform (interface server 150). Such mentorships may be desirable for specific types of surgeries such as minimally invasive surgery (MIS) as described below with examples.

### 4. Minimally Invasive Surgery

In an operating room setup of minimally invasive surgery (MIS) such as Laparoscopic surgery, the surgeon operates on a patient using surgical instruments inserted through small incisions. These surgical instruments can either be manually operated (such as laparoscopic instruments) or robotically actuated. Along with instruments, a scope (camera) is also inserted inside the patient's body to visualize the interaction of surgical instruments'tooltips with the tissue.

In the case of manual MIS, the surgeon directly controls the movements of the tooltips, whereas in the case of robotic MIS, the surgeon indirectly controls the movement of robotically actuated tooltips via an interface on the console. In both cases of MIS, the surgical field exhibits the complex interaction of highly articulated surgical instrument tooltips with the tissue to be operated.

With the current existing tele-mentoring technologies, an expert surgeon can assist the operating surgeon by providing guidance information in the form of either markings or hand gestures. However, this information is limited because of its two-dimensional and static nature. Current tele-mentoring still involves basic exchange of audio and annotated video messages, and lacks augmentation of information pertaining to surgical tool motion and tool-tissue interaction. As a result, it is difficult for the operating surgeon to visualize, comprehend and perform the required surgical tooltip movements.

As of now, whenever any surgeon does any complicated surgery for the first time, he/she decides some critical steps on the OT tables or sometime calls nearby surgeon friends. Due to this, most of the complication increases and even death of patients also happens. If complication in surgery happens with young surgeons, it breaks their confidence and that surgeon never takes complicated surgery and refers the cases to other surgeons. This approach never allows them to become a renowned and skilled surgeon.

Since it is not possible for the patient to travel large distance to get treatment from expert surgeon or expert surgeons to travel large distance for surgery, so a large chunk of patients is deprived of uncomplicated and risk-free surgery.

Interface server 150, provided according to several aspects of the present invention, provides a digital platform in which young surgeons can request any expert surgeon of their choice from across the globe, mentioning the patient's profile and complications and lab reports for paid mentorship during MIS such as laparoscopic surgery. The manner in which interface server 150 provides mentorship to surgical practitioners is described below with examples.

### 5. Providing Mentorship to Surgical Practitioners

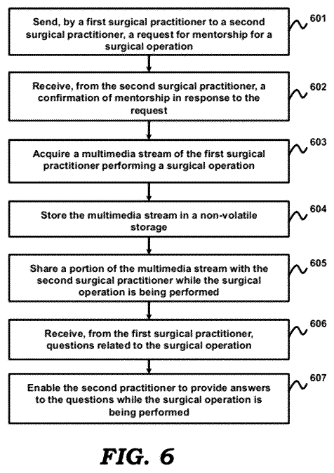

FIG. 6 is a flow chart illustrating the manner in which mentorship to surgical practitioners is provided according to aspects of the present invention. The flowchart is described with respect to FIG. 1, in particular, interface server 150, merely for illustration. However, various features can be implemented in other systems and/or other environments also without departing from the scope of various aspects of the present invention, as will be apparent to one skilled in the relevant arts by reading the disclosure provided herein.

In addition, some of the steps may be performed in a different sequence than that depicted below, as suited in the specific environment, as will be apparent to one skilled in the relevant arts. Many of such implementations are contemplated to be covered by several aspects of the present invention.

In step 601, interface server 150 sends, by a first surgical practitioner (e.g., junior surgeon) to a second surgical practitioner (e.g., senior surgeon), a request for mentorship for a surgical operation. The request may be sent in a known way (e.g., via email) and may include a profile of the patient to be operated upon, any complications that are expected during the surgical operation and lab reports related to the patient that may be of relevance during the surgical operation.

According to an aspect, interface server 150 maintains the details of one or more surgical practitioners and facilitates the first surgical practitioner to select the second surgical practitioner from the one or more surgical practitioners.

In step 602, interface server 150 receives, from the second surgical practitioner, a confirmation of mentorship in response to the request. The confirmation may also be received in a known way (e.g., via email).

In step 603, interface server 150 acquires a multimedia stream of the first surgical practitioner performing a surgical operation. According to an aspect, interface server 150 acquires the multimedia stream by displaying on an operation theater (OT) monitor, a video of the performance of the surgical operation captured using one or more cameras, recording the video being displayed on the OT monitor and converting the video to the multimedia stream. An example implementation of acquisition of a multimedia stream is described above with respect to FIG. 3.

In step 604, interface server 150 stores the multimedia stream in a non-volatile storage such as data store 180. The multimedia stream may be stored in any desirable format as will be apparent to one skilled in the arts.

In step 605, interface server 150 shares a portion of the multimedia stream with the second surgical practitioner while the surgical operation is being performed. Such sharing may entail streaming the portion of the multimedia stream via a network (such as 130) to one or more end user systems 110a-110c used by the one or more surgical practitioners. The one or more surgical practitioners to whom the multimedia stream is streamed may be determined in a known way.

In step 606, interface server 150 receives, from the first surgical practitioner, questions related to the surgical operation. The questions may be received as text, audio, or video or a combination thereof.

In step 607, interface server 150 enables the second practitioner to provide answers to the questions while the surgical operation is being performed. For example, questions may be received via audio from the first surgical practitioner while the surgical operation is being performed, with the second surgical practitioner then providing the answers via audio or text which are then provided to the first surgical practitioner.

Thus, interface server 150 (along with data store 180) provides a digital platform for telementorship. More particularly, it relates to a digital platform in which young surgeons can request from any expert surgeon of their choice, from across the globe, mentioning the patient profile, complications, and lab reports for paid telementorship during MSI such as laparoscopic surgery. In one embodiment, such a form of telementorship will be one to one interaction between mentor and mentee through the live surgery model described above.

The manner in which interface server 150 provides aspects of the present invention according to the operation of FIG. 6 is described below with examples.

### 6. Illustrative Example

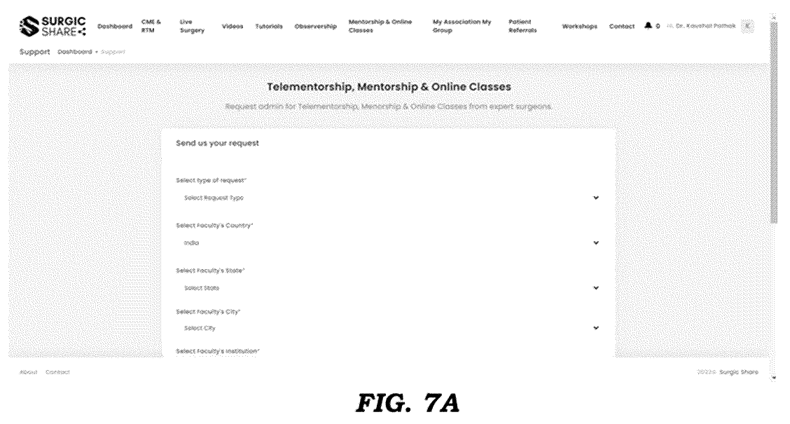

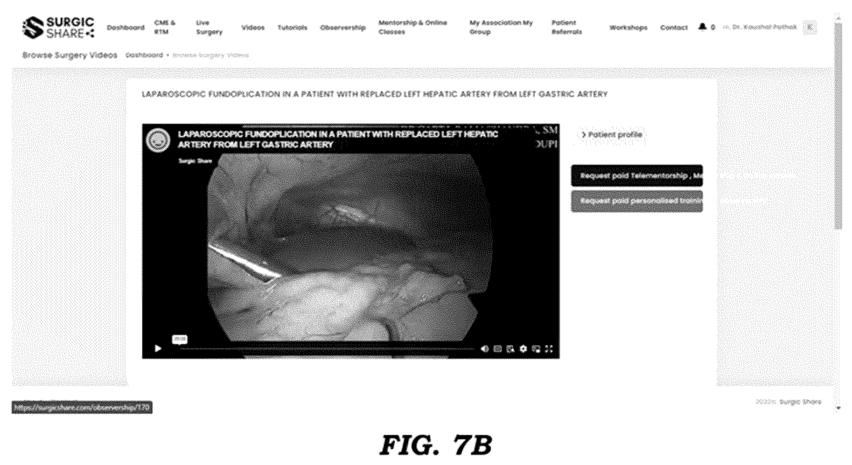

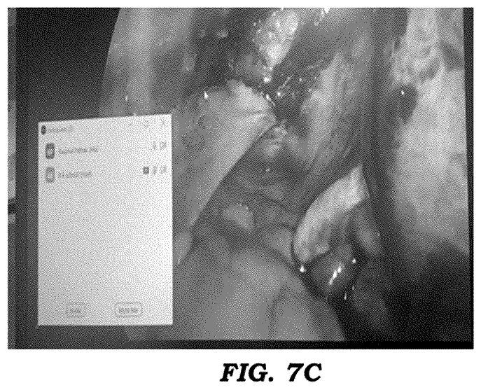

FIGS. 7A-7C together illustrate the manner in which a digital platform for telementorship is provided in one embodiment. Each of the Figures is described in detail below.

Referring again to FIG. 4, option 490 “Telementorship, Mentorship & Online Classes” facilitates a surgeon to request for a telementorship/mentorship from other surgeons. FIG. 7A illustrates a user interface using which a surgical practitioner is facilitated to request a mentorship in one embodiment. FIG. 7B illustrates a user interface using which a surgical practitioner is facilitated to request a mentorship in another embodiment. Specifically, the user interface there indicates that a junior surgeon requests mentorship after viewing a surgery video posted/uploaded by the desired mentor/senior surgeon.

FIG. 7C illustrates a user interface of live streaming of a surgical operation in progress as part of mentorship in one embodiment. It may be observed that that an audio channel has been enabled (indicate by the mike button) between a surgical practitioner performing the surgery (indicated as “Host”) and a surgical practitioner viewing the surgery (indicated as “Me”). By using the user interfaces of FIGS. 7A-7C, junior surgeons are enabled to request mentorship from senior surgeons and to take their assistance during live surgery.

It may be appreciated that senior surgeons can post his/her surgery videos, exhibiting his/her unique surgical skills. Since, before any particular complicated surgery, a junior surgeon can search for surgery videos of other surgeons for similar complicated case. He/she can request for telementorship from expert surgeon for his upcoming case, if he/she is satisfied with the unique surgical skills of expert surgeon.

At the time of surgery, operating/junior surgeon connects his OT monitor with a video capture device and then to a local system/laptop as a result of which, laptop displays same screen as of OT monitor. Then, he/she shares the screen with the mentor surgeon, and as such mentor surgeon can see same OT monitor screen same time as of operating surgeon. Therefore, the mentor surgeon can monitor all the movement of operating surgeon and guide him/her. As a result of which, chances of complications are very less. Even if complication happens, senior surgeon can give perfect solution at that point of time. This will improve confidence of operating surgeon and also the surgical outcome.

Thus, the present disclosure relates to a digital platform that allows senior surgeons to post and share live surgical videos. It gives an opportunity to the young aspirants and students to learn surgical skills at their own convenience sitting at home. More particularly, the digital platform provides access to the young surgeons and student to watch live surgical videos, tutorials, observership courses, research papers and publications. The young surgeons are facilitated to organize and attend live surgeries comfortably with saving of time, energy & money.

The digital platform contains user interfaces which are suitable for medical care services in particular. The user corresponds to the young surgeons and students aspiring for surgical specialty. Surgeons can create closed groups and conduct video conference meetings with group members so they can post and share live surgical videos.

The digital platform also enables senior surgeons to post upcoming observership course, along with the details of the course, fees, and the number of slots available for the course. Along with the course, users can access tutorials, research papers and publications. A closed group of surgeons can be formed where they can post and share surgery videos, tutorials and other courses which are helpful to the young aspirants or students in the domain.

The benefits of the digital platform include: Easy access to the observership course through registration; and User can request for mentorship, observership and online classes from the surgeons. The technology developed herein is novel in terms of its application and usage. Easy to create closed group of thousands of members within minutes. Organizing and attending Live surgery comfortably with saving of time, energy & money. All the surgeons can post their full surgery videos and tutorials in the closed group and other surgeon can see full surgery videos & tutorials depending on his/her availability.

Senior surgeons can post his/her observership course and demonstrate best products/technique without influence of any company. Good source of income for senior surgeon through observership course, as well. Junior surgeons can attend Observership course, request for mentorship, online classes from the dream surgeons and learn best surgical skills. Surgeons can post upcoming hands-on training course and junior surgeons and students can register for the course.

It should be appreciated that the above noted features can be implemented in various embodiments as a desired combination of one or more of hardware, execution modules and firmware. The description is continued with respect to one embodiment in which various features are operative when execution modules are executed.

### 7. Digital Processing System

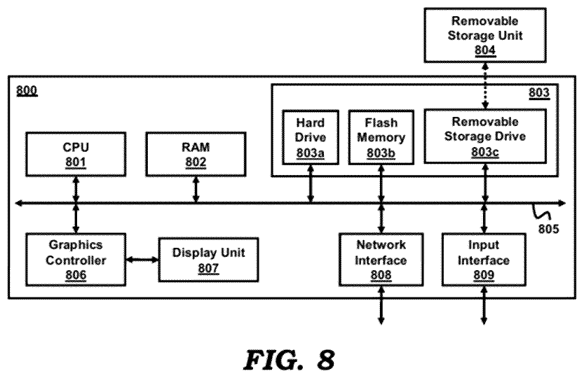

FIG. 8 is a block diagram illustrating the details of digital processing system 800 in which various aspects of the present invention are operative by execution of appropriate execution modules. Digital processing system 800 may correspond to interface server 150 or end user systems 110a-110c.

Digital processing system 800 may contain one or more processors (such as a central processing unit (CPU) 801), random access memory (RAM) 802, secondary memory 803, graphics controller 806, display unit 807, network interface 808, and input interface 809. All the components except display unit 807 may communicate with each other over communication path 805 which may contain several buses as is well known in the relevant arts. The components of FIG. 8 are described below in further detail.

CPU 801 may execute instructions stored in RAM 802 to provide several features of the present invention. CPU 801 may contain multiple processing units, with each processing unit potentially being designed for a specific task. Alternatively, CPU 801 may contain only a single general-purpose processing unit. RAM 802 may receive instructions from secondary memory 803 using communication path 805.

Graphics controller 806 generates display signals (e.g., in RGB format) to display unit 807 based on data/instructions received from CPU 801. Display unit 807 contains a display screen to display the images defined by the display signals (e.g., portions of the user interfaces shown in FIGS. 4, 5A-5I and 7A-7C). Input interface 809 may correspond to a keyboard and a pointing device (e.g., touch-pad, mouse), which enable the various inputs to be provided (e.g., inputs for the user interfaces shown in FIGS. 4, 5A-5I and 7A-7C).

Network interface 808 provides connectivity to a network (e.g., using Internet Protocol), and may be used to communicate with other connected systems. Network interface 808 may provide such connectivity over a wire (in the case of TCP/IP based communication) or wirelessly (in the case of WIFI, Bluetooth based communication).

Secondary memory 803 may contain hard drive 803a, flash memory 803b, and removable storage drive 803c. Secondary memory 803 may store the data (e.g., portions of the data provided as inputs in the user interfaces shown in FIGS. 4, 5A-5I and 7A-7C) and software instructions (e.g., for implementing the steps of FIGS. 2 and 6, the modules and systems of FIG. 3), which enable digital processing system 800 to provide several features in accordance with the present invention.

Some or all of the data and instructions may be provided on removable storage unit 804, and the data and instructions may be read and provided by removable storage drive 803c to CPU 801. Floppy drive, magnetic tape drive, CD-ROM drive, DVD Drive, Flash memory, removable memory chip (PCMCIA Card, EPROM) are examples of such removable storage drive 803c.

Removable storage unit 804 may be implemented using storage format compatible with removable storage drive 803c such that removable storage drive 63c can read the data and instructions. Thus, removable storage unit 804 includes a computer readable storage medium having stored therein computer software (in the form of execution modules) and/or data.

However, the computer (or machine, in general) readable storage medium can be in other forms (e.g., non-removable, random access, etc.). These “computer program products” are means for providing execution modules to digital processing system 800. CPU 801 may retrieve the software instructions (forming the execution modules) and execute the instructions to provide various features of the present invention described above.

It should be understood that the figures and/or screen shots shown above highlighting the functionality and advantages of the present invention are presented for example purposes only. The present invention is sufficiently flexible and configurable, such that it may be utilized in ways other than that shown in the figures.

Merely for illustration, only representative number/type of graph, chart, block, and sub-block diagrams were shown. Many environments often contain many more block and sub-block diagrams or systems and sub-systems, both in number and type, depending on the purpose for which the environment is designed.

While specific embodiments of the invention have been shown and described in detail to illustrate the inventive principles, it will be understood that the invention may be embodied otherwise without departing from such principles.

It should be understood that the examples and embodiments described herein are for illustrative purposes only and that various modifications or changes in light thereof will be suggested to persons skilled in the art and are to be included within the spirit and purview of this application and scope of the appended claims.

I/We claim:

1. A method of providing a digital platform for surgical practitioners, the method comprising:
acquiring a multimedia stream of a first surgical practitioner performing a surgical operation;
storing the multimedia stream in a non-volatile storage;
sharing a portion of the multimedia stream with one or more surgical practitioners while the surgical operation is being performed;
receiving, from a second surgical practitioner, questions related to the surgical operation; and
enabling the first surgical practitioner to provide answers to the questions while the surgical operation is being performed.

2. The method of claim 1, wherein the acquiring comprises:
displaying on an operation theater (OT) monitor, a video of the performance of the surgical operation captured using one or more cameras;
recording the video being displayed on the OT monitor; and
converting the video to the multimedia stream,
wherein the sharing comprises streaming the portion of the multimedia stream via a network.

3. The method of claim 1, comprising:
uploading, by the first surgical practitioner, portions of the multimedia stream as corresponding videos; and
facilitating the second surgical practitioner to view the corresponding videos.

4. The method of claim 3, wherein each video is associated with one or more of tutorials, research papers and publications.

5. The method of claim 1, comprising:
posting, by the first surgical practitioner prior to the surgical operation, an observership course;
determining that a set of surgical practitioners have registered for the observership course; and
including the set of surgical practitioners in the one or more surgical practitioners whereby the set of surgical practitioners are enabled to observe and interact with the first surgical practitioner while the surgical operation is being performed.

6. The method of claim 1, comprising:
creating a group of surgical practitioners; and
sharing the portion of the multimedia stream with the group of surgical practitioners while the surgical operation is being performed.

7. A digital platform for surgical practitioners, the digital platform comprising:
an acquisition system to acquire a multimedia stream of a first surgical practitioner performing a surgical operation and to store the multimedia stream in a non-volatile storage;
a streaming server to share a portion of the multimedia stream to one or more surgical practitioners while the surgical operation is being performed; and
an interface system to receive from a second surgical practitioner, questions related to the surgical operation and to enable the first practitioner to provide answers to the questions while the surgical operation is being performed.

8. The digital platform of claim 7, wherein the acquisition system comprises:
an operation theater (OT) monitor to display a video of the performance of the surgical operation captured using one or more cameras;
a recorder to record the video being displayed on the OT monitor; and
a converter to convert the video to the multimedia stream,
wherein to share, the streaming server streams the portion of the multimedia stream via a network.

9. The digital platform of claim 7, wherein the interface system is operable to:
upload, by the first surgical practitioner, portions of the multimedia stream as corresponding videos; and
facilitate the second surgical practitioner to view the corresponding videos.

10. The digital platform of claim 9, wherein each video is associated with one or more of tutorials, research papers and publications.

11. The digital platform of claim 7, wherein the interface system is operable to:
post, by the first surgical practitioner prior to the surgical operation, an observership course;
determine that a set of surgical practitioners have registered for the observership course; and
include the set of surgical practitioners in the one or more surgical practitioners to whom the portion of the multimedia stream is streamed.

12. The digital platform of claim 7, wherein the interface system is operable to:
create a group of surgical practitioners; and
share the portion of the multimedia stream with the group of surgical practitioners while the surgical operation is being performed.

13. A non-transitory machine-readable medium storing one or more sequences of instructions for providing a digital platform for surgical practitioners, wherein execution of the one or more instructions by one or more processors causes a digital processing system to perform the actions of:
acquiring a multimedia stream of a first surgical practitioner performing a surgical operation;
storing the multimedia stream in a non-volatile storage;
sharing a portion of the multimedia stream with one or more surgical practitioners while the surgical operation is being performed;
receiving, from a second surgical practitioner, questions related to the surgical operation; and
enabling the first practitioner to provide answers to the questions while the surgical operation is being performed.

14. The non-transitory machine-readable medium of claim 13, wherein the acquiring comprises one or more instructions for:
displaying on an operation theater (OT) monitor, a video of the performance of the surgical operation captured using one or more cameras;
recording the video being displayed on the OT monitor; and
converting the video to the multimedia stream,
wherein the sharing comprises streaming the portion of the multimedia stream via a network.

15. The non-transitory machine-readable medium of claim 13, comprising one or more instructions for:
uploading, by the first surgical practitioner, portions of the multimedia stream as corresponding videos; and
facilitating the second surgical practitioner to view the corresponding videos.

16. The non-transitory machine-readable medium of claim 15, wherein each video is associated with one or more of tutorials, research papers and publications.

17. The non-transitory machine-readable medium of claim 13, comprising one or more instructions for:
posting, by the first surgical practitioner prior to the surgical operation, an observership course;
determining that a set of surgical practitioners have registered for the observership course; and
including the set of surgical practitioners in the one or more surgical practitioners whereby the set of surgical practitioners are enabled to observe and interact with the first surgical practitioner while the surgical operation is being performed.

18. The non-transitory machine-readable medium of claim 13, comprising one or more instructions for:
creating a group of surgical practitioners; and
sharing the portion of the multimedia stream with the group of surgical practitioners while the surgical operation is being performed.
